(19) United States
(12) Patent Application Publication
(10) Pub. No.: US 2022/0071781 A1
(43) Pub. Date: Mar. 10, 2022

(54) LINER FOR AN AMPUTATION STUMP

(71) Applicant: CINOGY GMBH, Duderstadt (DE)

(72) Inventors: Leonhard Trutwig, Duderstadt (DE); Dirk Wandke, Heilbad Heiligenstadt (DE); Karl-Otto Storck, Duderstadt (DE); Mirko Hanhl, Berlingerode (DE); Melanie Ricke, Katlenburg-Lindau (DE); Marcel Jung, Duderstadt (DE); Klaus Johannes, Gleichen (DE); Martin Hillmann, Duderstadt (DE)

(21) Appl. No.: 17/413,637

(22) PCT Filed: Dec. 18, 2019
(86) PCT No.: PCT/DE2019/101104

§ 371 Date: Aug. 20, 2021

(30) Foreign Application Priority Data
Dec. 19, 2018 (DE) 10 2018 132 918.0

(51) Int. Cl. A61F 2/78; H05H 1/24

(52) U.S. Cl. CPC A61F 2/7812; H05H 2245/30; A61F 2002/7818; H05H 1/2418

(57) ABSTRACT

A liner for application to an amputation stump as a cushion, having a proximal entry opening (2) of a wall (6) having a sleeve section (3) provided for circumferentially enclosing the amputation stump, and a distal closed end section (4), wherein an inside (7) of the wall (6) of the liner (1) is designed for contact on the amputation stump, permits simplified handling of the wound care on the amputation stump of a prosthesis wearer in that an electrode arrangement (10), having at least one electrode (12), for a dielectric barrier plasma discharge is integrated into the liner (1), which arrangement extends starting from the distal end section (4) into the sleeve section (3), is connected at the distal end section (4) to at least one terminal (11) for a high-voltage control signal and is provided with a dielectric cover for contact on the amputation stump, and in that the dielectric cover is provided, at least in the region of the electrode arrangement (10) on the inside (7), with protrusions (8), which define at least one gas space (9, 9′) upon contact on the amputation stump, in which the dielectric barrier plasma discharge can form.

The invention relates to a liner for application to an amputation stump as a cushion, having a proximal entry opening, a wall having a sleeve section provided for the circumferentially enclosing amputation stump, and a distal closed end section, wherein an inside of the wall of the liner is designed for contact on the amputation stump.

Such liners are known in numerous embodiments. They typically consist of an elastic plastic material, such as polyurethane or silicone, and are formed having a wall thickness such that a cushioning effect occurs. Due to the elasticity thereof, the liners are to abut the amputation stump closely and in this way are to form a cushioning intermediate layer between the liner and a prosthesis socket, on which a prosthesis for an amputated limb is fastened. The system made up of prosthesis socket and liner is thus used for fastening a prosthesis on the amputation stump. This fastening can be assisted in a known technology in that a negative pressure is generated between liner and amputation stump and/or between liner and prosthetic socket, by which negative pressure the respective friction combination is reinforced.

An amputation stump occurs upon an amputation of an external limb and has at least one central bone, surrounding soft tissue, and an outer skin region. The closed skin layer present in the healthy body is interrupted by the amputation operation and initially forms—possibly together with the soft tissue—a wound surface and later forms a scar tissue. Even when the original amputation wound is healed, wound surfaces sometimes are formed on the amputation stump as the prosthesis is worn, which wound surfaces can arise due to pressure and abrasion points on the skin of the amputation stump for various reasons. In order that wearing a prosthesis, which significantly enhances the quality of life, is not prevented by a wound on the amputation stump, a wound of an amputation stump has to be cared for and caused to heal as quickly as possible. This is carried out by a treatment of the wounds using wound dressings and disinfecting and healing-promoting active ingredients, which are preferably applied in rest times after the prosthesis is removed.

It is fundamentally known that a wound treatment for the accelerated healing of a wound can also be carried out with the aid of a dielectric barrier plasma, which represents a so-called “cold plasma”. The ionization processes initiated by a high-frequency high-voltage alternating field cause reactive species to arise in the air, which have a germicidal effect and promote the microcirculation of the skin in the treated region. Therefore, planar wound dressings having a planar electrode arrangement are known for wound care, using which a dielectric barrier plasma can be generated on a skin surface for the purpose of wound healing. Adapting the contact area to the size of the wound is already known, so that a wound on an amputation stump could also be cared for in this way. The removal of the prosthesis is a requirement here. In particular for active prosthesis wearers, who do not wish to dispense with the prosthesis over long periods during the day, only a limited time possibility remains for the wound treatment.

The invention is based on the object of enabling simplified handling of the wound care on the amputation stump of a prosthesis wearer.

To achieve this object, according to the invention, a liner of the type mentioned at the outset is characterized in that an electrode arrangement including at least one electrode for a dielectric barrier plasma discharge is integrated into the liner, which arrangement extends starting from the distal end section into the sleeve section, is connected at the distal end section to at least one terminal for a high-voltage control signal, and is provided with a dielectric cover for contact on the amputation stump, and in that the dielectric cover is provided at least in the region of the electrode arrangement on the inside with protrusions, which define at least one gas space upon contact on the amputation stump, in which the dielectric barrier plasma discharge can form.

An electrode arrangement for the formation of a dielectric barrier plasma in the distal end region of the amputation stump is thus integrated in the liner according to the invention so that a wound treatment is possible by means of the dielectric barrier plasma discharge with attached prosthesis. In this way, not only is the possibility improved of performing a wound treatment frequently, if necessary, but the possibility is also opened up of performing a preventative treatment of the skin of the amputation stump, for example, by the displacement of the microcirculation in the affected tissue, to contain the formation of larger wound areas already as they occur. The electrode arrangement is located according to the invention where the most sensitive points are present on the amputation stump, namely at the distal end, thus where the severing of the amputated limb has taken place, and extending terminating thereon proximally into the sleeve section of the liner. It will generally be sufficient here if the electrode arrangement extends only over a part of the length of the sleeve section, even if a point susceptible to skin irritations is still located at the proximal edge of the prosthesis socket, which comes to rest in the region of the proximal end of the liner. In one embodiment of the liner, the electrodes can therefore also extend close to the proximal edge of the liner.

In one preferred embodiment of the liner, the dielectric cover of the electrode arrangement is formed by the wall of the liner itself. This is possible if the wall of the liner itself has sufficient dielectric properties, as can be the case, for example, with a silicone liner, or also with a liner made of a polyurethane gel. A typical production process for such a liner provides a casting procedure. The integration of the electrode arrangement into the wall of the liner can therefore take place during this casting procedure for producing the wall of the liner, by introducing the electrode arrangement in a suitable manner into the wall of the liner. It can be expedient for precise positioning of the electrode arrangement to design the casting procedure in two steps thus, for example, first to cast an outer layer of the wall of the liner, wherein the electrode arrangement can be applied on the inside to the cast first layer. A further layer having defined thickness is then produced by casting to complete the wall. In this way, the electrode arrangement is positioned in a defined manner, so that a sufficiently thick dielectric layer can be ensured between the electrode arrangement and the skin of the amputation stump, which reliably prevents a flashover of the high voltage to the amputation stump, which is painful and destroys tissue.

In one embodiment of the invention, the electrode arrangement extends starting from the distal terminal with finger-like electrode sections into the sleeve section of the liner, so that electrode sections are arranged respectively dielectrically separated from one another in the circumferential direction, at preferably equal intervals to one another.

The electrode arrangement can include two distal terminals, which are each connected to at least one electrode, wherein the electrodes are electrically insulated from one another. It is possible here, for example, to supply AC high voltages equal in amplitude and frequency to the two electrodes, which are shifted in relation to one another by half a period, so that the voltages completely compensate one another upon a superposition. The two electrodes can also have electrode sections extending like fingers here and can extend, for example, approximately over half a circumference of the liner.

It is reasonable for manufacturing if the protrusions on the inside of the wall are ribs aligned in the longitudinal direction. In this embodiment, a core of the casting mold defining the inner wall can be pulled without problems out of an outer mold when the material of the wall has cooled and cured from the liquid starting material to form the elastic wall material.

It can be expedient to produce a distal end region of the liner together with the electrode arrangement and the protrusions as a separate part and to connect it by way of a separately produced part, which represents a significant length of the sleeve section and the entry opening, to the distal part by welding, adhesive bonding, or the like. In this way, it is possible to produce a distal end part of the liner, which is complex to produce, and to connect it to different parts which determine the length of the sleeve section, in order to thus produce required liners of different lengths with identical distal end pieces using the simple completion parts.

The supply of the high voltage signal to the electrode arrangement can be carried out in one embodiment by means of a plug connection insertable in a locking manner into the liner. Magnets attracting one another can be provided to assist the contacting.

The invention is to be explained in greater detail hereinafter on the basis of exemplary embodiments illustrated in the drawing. In the figures:

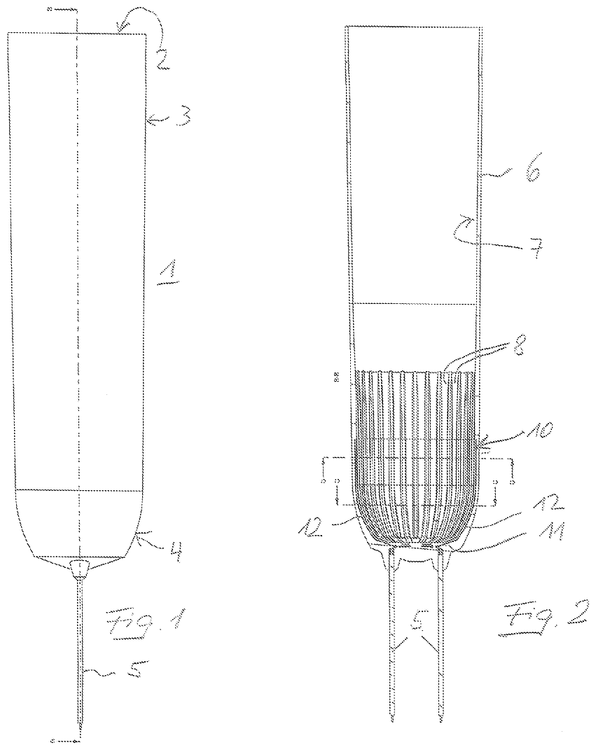

FIG. 1 shows a view of a liner according to the invention

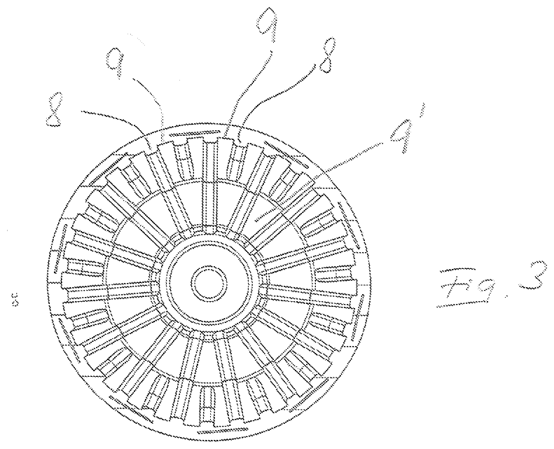

FIG. 2 shows a longitudinal section through the liner according to FIG. 1 along section line B-B

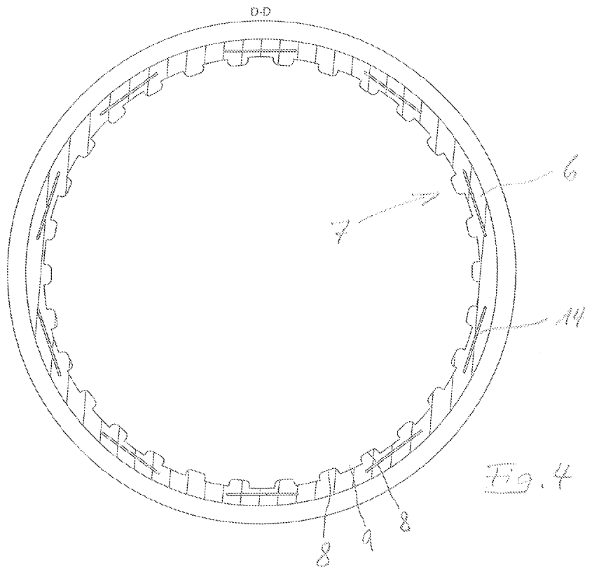

FIG. 3 shows a horizontal section through the liner along section line C-C from FIG. 2 with a view looking in the distal direction

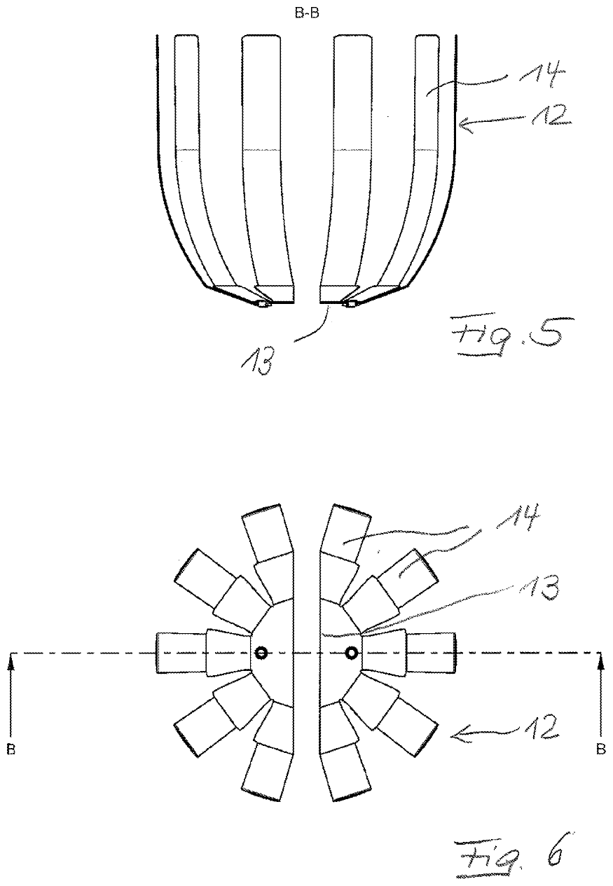

FIG. 4 shows an enlarged section through the liner according to FIG. 2 along section line D-D with a view looking in the proximal direction

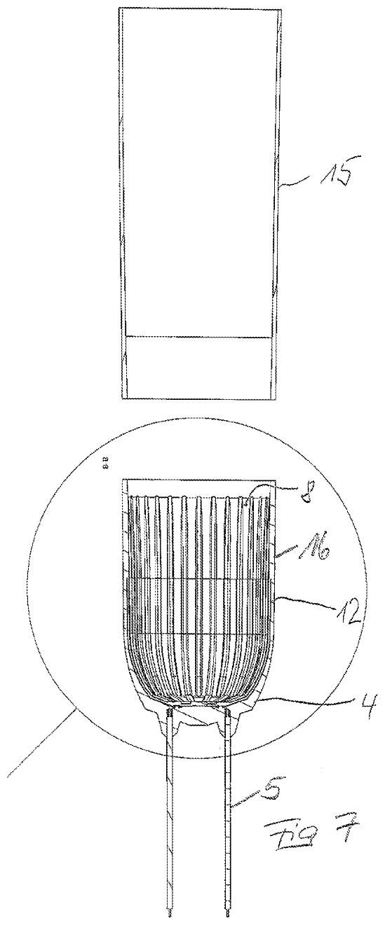

FIG. 5 shows a sectional illustration along line B-B in FIG. 6 of an electrode arrangement for the liner

FIG. 6 shows a schematic illustration of the electrode arrangement in a frontal view of the distal end of the liner

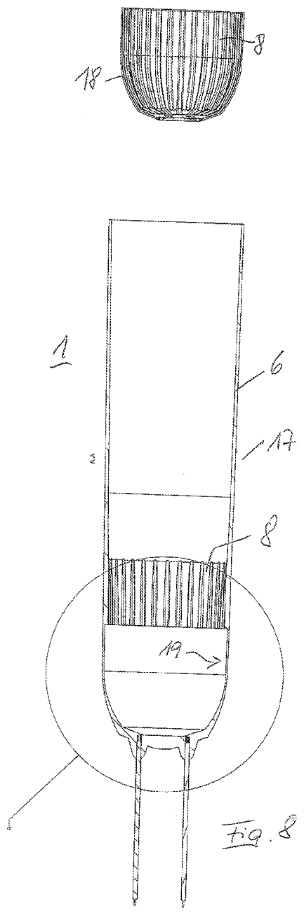

FIG. 7 shows a sectional illustration corresponding to FIG. 2 of a further embodiment of a liner according to the invention

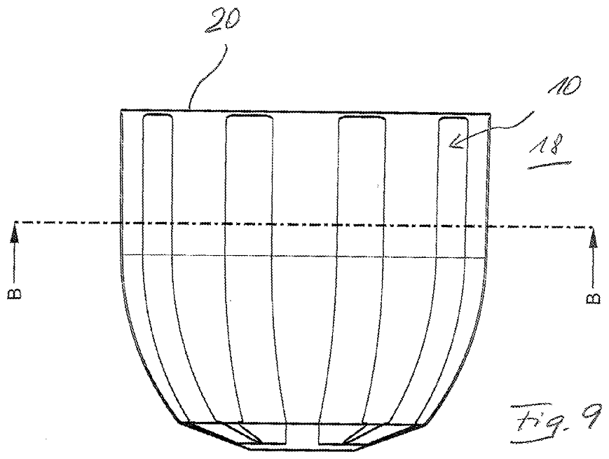

FIG. 8 shows a sectional illustration similar to FIG. 2 for a third embodiment of a liner having an insert bearing the electrode arrangement

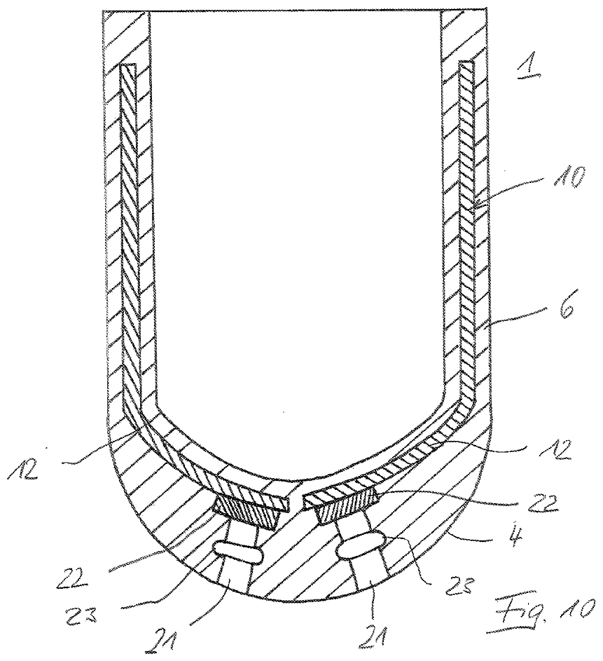

FIG. 9 shows a schematic view of the insert bearing the electrode arrangement

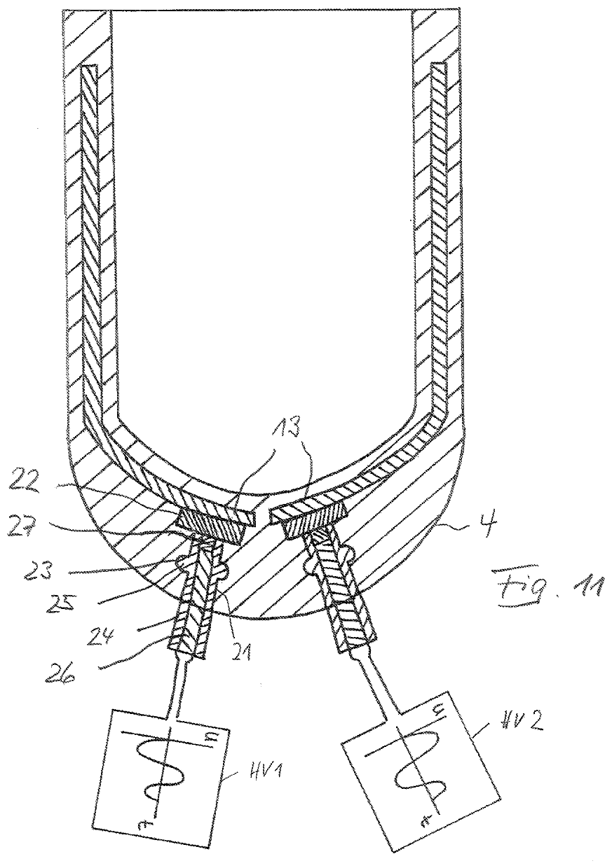

FIG. 10 shows a schematic sectional illustration of a distal end section of a liner according to a further embodiment having receptacle channels for high-voltage supply lines

FIG. 11 shows a schematic illustration according to FIG. 10 having coupling parts inserted into the liner for high-voltage supply lines.

FIG. 1 illustrates the typical form of a liner 1, which has a proximal entry opening 2 in a vertical longitudinal direction on the upper end, on which opening a sleeve section 3 adjoins distally. The sleeve section 3 is formed slightly conically in the illustrated exemplary embodiment, so that the liner 1 tapers slightly distally. The sleeve section 3 emerges at the distal end into a closed end section 4, from which two connecting cables 5 protrude. The closed distal end section 4 tapers in rounded shape and thus forms a distal terminus of the liner 1 known per se.

The sectional illustration of FIG. 2 shows that the illustrated exemplary embodiment of a liner 1 has a one-piece wall 6, which is formed from an elastic, cushioning material, having a wall thickness such that it forms a good cushion for an amputation stump (not shown), on which the liner 1 is applied. The application of the liner 1 can be carried out in a known way by pulling onto the amputation stump or in that the wall 6 of the liner 1 is rolled up from the entry opening 2 to the distal end and after the insertion of the amputation stump into the rolled-up liner 1, is unrolled onto the outside of the amputation stump. In the last-mentioned application method of the liner 1, the wall 6 can also be made somewhat adhesive on its inside 7.

The liner 1, which is insofar conventionally constructed, is provided according to the invention at the distal end with an inside 7 of the wall 6 equipped with longitudinal ribs 8. The longitudinal ribs 8 extend in a star shape in the distal end section 4. To be able to arrange the longitudinal ribs 8 close enough to one another, long and short longitudinal ribs 8 are alternately arranged adjacent to one another, so that the long longitudinal ribs 8 extend farther into the distal end section 4 than the adjacent short longitudinal ribs 8, as is illustrated in particular in FIG. 3.

The liner 1 abuts the amputation stump at the distal end provided with the longitudinal ribs 8 via the longitudinal ribs 8. The longitudinal grooves 9 located between the longitudinal ribs 8 thus form chambers abutting to the amputation stump, in which air is located. Corresponding chambers 9′ result in the region of the distal end section, where only long longitudinal ribs 8 are still located, in the intermediate space between the longitudinal ribs 8.

As may be seen from FIGS. 2 and 3, an electrode arrangement 10 is embedded in the wall 6 of the liner 1, which arrangement extends from at least one distal terminal 11 in the distal end section 4 into the sleeve section 3. The electrode arrangement 10 is thus a planar arrangement inside the wall 6 of the liner 1. In the illustrated exemplary embodiment, the electrode arrangement 10 consists of two electrodes 12, which are insulated from one another and protrude starting from a distal base part 13 with finger-like electrode sections 14 into the sleeve section 3 (cf. FIG. 5 and FIG. 6). It is recognizable that the longitudinal ribs 8 are formed longer than the electrode sections 14.

High-frequency AC voltage signals are connectable to the connecting cables 5, which signals are applied to the electrodes 12 as high-frequency high-voltage alternating potentials. In principle, the two electrodes 12 can be supplied in a typical way from an AC voltage source, so that one of the two electrodes 12 functions as a reference or ground electrode and the other electrode is subjected to potentials periodically changing in their polarity. However, it is preferred if the two electrodes 12 are each supplied from an AC voltage source with AC voltage potentials opposite in the frequency and in the amplitude thereof, the average potential of which is a ground potential. In this arrangement, the amputation stump functions as the counter electrode to the two electrodes 12. The air located in the longitudinal grooves 9 and in the chambers 9′ is ionized by the AC voltage field generated by the electrodes 12, so that with suitable activation, a plasma can form, which acts on the surface of the amputation stump and has a healing-promoting or preventative effect there both due to disinfection and also due to the increase of microcirculation in the tissue.

The activation of the electrodes 12 using the high-frequency high-voltage AC signals is known to a person skilled in the art. The AC voltage signals can have a harmonic wave function, but are preferably pulsed signals having alternating polarity in relation to the reference potential, wherein the peak voltage of the pulses can be between two and forty kV and the AC voltage frequency can be between several 100 kHz to several 100 MHz. In special cases, the excitation frequency can even go into the GHz range.

If opposite and equal activation signals are used, in principle the activation signals cancel out completely at equal distance from the two electrodes 12. Such an activation therefore has significant advantages for the electromagnetic compatibility in the far range of the electrodes 12. At close range, the advantage arises that the potential difference between the electrodes 12 is twice as high as with a typical single activation from an AC voltage source having an alternating potential and a reference potential.

It may be seen from FIG. 4 that the finger-like electrode sections 14 are arranged at approximately equal intervals to one another on the circumference of the wall 6. In this way, a uniform supply with a plasma field may be achieved over the circumference of the liner 1—and thus of the amputation stump. The longitudinal ribs 8 are used as spacers when they abut the surface of the amputation stump as the inside 7 of the liner 1. The longitudinal grooves 9 between the longitudinal ribs 8 contain the air in which the plasma can form upon application of the high-voltage control signals at the electrodes 12, so that the plasma is in direct contact with the surface of the amputation stump.

It is essential for the formation of a dielectric barrier plasma in the longitudinal grooves 9 or in chambers 9′ that the electrodes 12 are completely shielded by a dielectric material from the amputation stump in the interior of the liner 1 so that electric arcs cannot form between the electrodes 12 and the amputation stump. The direct current flow has to be reliably prevented by the dielectric material. In the exemplary embodiment shown in FIGS. 1 to 6, the wall 6 in which the electrodes 12 are introduced functions as the dielectric material. The condition for this embodiment is thus that the material of the wall 6 of the liner 1 has sufficiently good dielectric properties. This is readily achievable, for example, in silicone liners and polyurethane liners by a corresponding selection of the material.

The exemplary embodiment shown in FIG. 7 differs from the embodiment, described up to this point, of a liner 1 according to the invention in that the wall 6 of the liner 1 consists of two parts. A proximal sleeve part 15 is constructed as a simple cylindrical or slightly conically tapering sleeve part.

It is connectable, for example, by adhesive bonding or welding to a distal end part 16, in which the longitudinal ribs 8 and the electrode arrangement 10 are located with the distal end section 4 and the connecting cables 5. The distal end part 16 therefore contains all functions for forming the dielectric barrier plasma, while the proximal sleeve part 15 only determines the total length of the liner 1. Since liners 1 of different lengths are required for different amputations and different body sizes, the distal end part 16 can be manufactured uniformly and can merely be combined with proximal sleeve parts of different lengths to form liners 1 of differing total length.

Another exemplary embodiment of a liner 1 according to the invention is illustrated in FIGS. 8 and 9. In this case, the liner 1 consists of a liner shell 17 and an insert part 18. The liner shell 17 forms the wall 6 of the liner 1 and in the illustrated exemplary embodiment contains a proximal part of the longitudinal ribs 8 or the longitudinal grooves 9. Toward the distal end section 4, the wall 6 of the liner shell 17 is formed thin and unprofiled and thus forms a recess 19 in the size of the insert part 18. The insert part 18 contains the remaining longitudinal ribs 8 and longitudinal grooves 9 and also chambers 9′ and the electrode arrangement 10 having the terminals 11 for the connecting cables 5.

The insert part 18 bears the electrode arrangement 10 on the outside and is terminated toward the inside 7 of the liner 1 by a completely closed dielectric material 20, which is only open on top. The electrode arrangement 10 is constructed in principally the same way as explained in particular on the basis of FIGS. 5 and 6. The dielectric material 20 here forms the inside 7 of the wall 6 having the longitudinal ribs 8 and the longitudinal grooves 9 as well as the chambers 9′. This embodiment offers manufacturing advantages, since the functional parts of the liner 1 can be implemented practically completely in the insert part 18, on which the electrode arrangement 10 solely has to be applied on the outside. After the insertion of the insert part 18 into the liner shell 17, the electrode arrangement 10 is protected against a touch from the outside by the wall of the liner shell 17.

Of course, it is conceivable in a further variant to form the liner shell 17 without longitudinal ribs 8 and to provide it on the inside 7 only with a step to form the recess 19, into which the insert part 18 can be inserted. In this case, in contrast to the illustrated embodiment, the insert part 18 would contain the complete longitudinal ribs 8. The use of the insert part 18 can furthermore offer the advantage that the dielectric material 20 used for the insert part 18 is selected as a special material, which can differ from the wall material of the liner shell 17.

It is thus possible to form the wall 6 of the liner shell 17 particularly flexibly, for example, in order to simplify the rolling of the liner 1 onto the amputation stump, while the insert part 18 quasi-functions as a distal end cap, which can be formed having a reduced elasticity, but better dielectric properties.

A further embodiment of a liner 1 according to the invention is shown in FIGS. 10 and 11, which essentially corresponds to the embodiment according to FIG. 1, wherein the electrode arrangement 10 is embedded in the wall 6 of the liner 1. In contrast to the embodiment according to FIGS. 1 to 3, the two electrodes 12 of the electrode arrangement 10 are not connected to connecting cables 5, which protrude out of the distal closed end section 4. Instead, two channels 21 are provided in the distal end section 4, which are open to the outside and are each terminated toward the electrodes 12 by a permanent magnet 22. The permanent magnets 22 are electrically conductive and each abut one of the electrodes 12, so that each permanent magnet 22 is electrically connected to one of the electrodes 12. A ring-shaped locking recess 23 is provided in each channel 21. FIG. 11 illustrates that a correspondingly shaped contact part 24 is inserted into the channel 21 having its ring-shaped locking recess 23, which contact part engages with a ring-shaped locking bead 25 in the ring-shaped locking recess 23 and is thus inserted in a locking manner into the channel 21. The contact part 24 consists of an electrically insulating material, which encloses a central conductor 26 in an insulating manner. The central conductor 26 is connected at the end face to a counter magnet 27, which is formed reversed in polarity to the permanent magnet 22 of the channel 21, so that permanent magnet 22 and counter magnet 27 attract one another and thus establish a reliable electrical connection between the conductor 26 and the associated electrode 12.

It is schematically indicated in FIG. 11 that the central conductor 26 is respectively connected to a high-voltage source HV1, HV2, wherein the connection is protected in a way known to a person skilled in the art against touching and high voltage flashovers, of course. It can be schematically seen that the two high-voltage sources HV1 and HV2 emit high-voltage signals, which are opposite and equal, so that ideally a total signal of 0 results. The two signals are thus equal in frequency and each have an oppositely oriented amplitude. The signals are preferably pulsed, wherein each pulse has the form of a damped oscillation, thus an oscillation having a rapidly decaying amplitude.

The opposite and equal high voltages of the two voltage sources HV1 and HV2 are suitable for generating a close plasma field. However, the high-voltage fields compensate one another with greater distance to the electrodes 12, so that at some distance relevant electromagnetic disturbance due to the fields initiating the plasma no longer exists.

Of course, the effect using the two high-voltage sources HV1 and HV2 can be dispensed with. It is readily possible to form the electrode arrangement 10 as a single electrode, for which the amputation stump forms the counter electrode, so that an intensive plasma field treatment using a dielectric barrier plasma is enabled on the skin and possibly wound points of the amputation stump. In this case, the formation of a single channel 21 in the distal end section 4 of the liner 1 is sufficient.

The two high-voltage sources HV1 and HV2 can be formed in the same voltage supply, for example, using two transformers wound in opposite directions, the primary coils of which can be activated using the same control signals. The two transformers can possibly also be formed by secondary windings, which—insulated from one another—are wound on the same primary winding. One possible embodiment is that the two secondary windings are arranged adjacent to one another in the axial direction on one oblong primary winding. Of course, it is also possible to activate two separate transformers having separate primary and secondary windings using the same control signals to form the two high-voltage sources HV1 and HV2.

The embodiment illustrated in FIGS. 10 and 11 has the advantage that the contacting of the high-voltage line does not have to be carried out during the production of the liner. The contacting takes place via the contact part 24, which is inserted in a locking manner into the channel to connect the high-voltage source HV1, HV2 to an electrode 12 using a central conductor 26. The contact part 24 forms a locking plug connection together with the channel, by which the contacting with the electrode 12 can already be established. In the illustrated exemplary embodiment, the contacting is thus improved and made more secure in that the electrode 12 is provided with the permanent magnet 22 in that, for example, the permanent magnet 22 is adhesively bonded via a conductive adhesive on the base part 13 of the electrode 12. The contacting is also ensured within the flexible material of the liner 1 by the magnetic attraction force active between the permanent magnet 22 and the counter magnet 27, even if the material of the liner has a certain instability and stabilization measures are to be dispensed with.
1 liner
2 entry opening
3 sleeve section
4 end section
5 connecting cable
6 wall
7 inside (of 6)
8 longitudinal ribs
9 longitudinal grooves
9′ chambers
10 electrode arrangement
11 terminal
12 electrodes
13 base part
14 finger-like electrode sections
15 sleeve part
16 distal end part
17 liner shell
18 insert part
19 recess
20 dielectric material
21 channels
22 permanent magnets
23 ring-shaped locking recess
24 contact part
25 ring-shaped locking bead
26 central conductor
27 counter magnet

1. A liner for application to an amputation stump as a cushion, comprising:
a wall having a sleeve section provided for circumferentially enclosing the amputation stump, wherein the wall has a proximal entry opening and a distal closed end section, wherein an inside of the wall is designed for contact on the amputation stump;
an electrode arrangement comprising at least one electrode for a dielectric barrier plasma discharge integrated into the liner and which extends from the distal closed end section into the sleeve section;
at least one terminal for a high-voltage control signal connected to the electrode arrangement at the distal closed end section; and
a dielectric cover for the electrode arrangement which contacts the amputation stump, wherein the dielectric cover may be separate from or part of the wall, and wherein the dielectric cover comprises at least in a portion of a region of the electrode arrangement on the inside of the wall protrusions which define at least one gas space upon contact with the amputation stump in which the dielectric barrier plasma discharge forms.

2. The liner as claimed in claim 1, wherein the dielectric cover of the electrode arrangement is formed by the wall of the liner.

3. The liner as claimed in claim 1 wherein the electrode arrangement extends from the at least one terminal with finger-like electrode sections into the sleeve section of the liner.

4. The liner as claimed in claim 1 wherein the electrode arrangement extends only over a part of a length of the sleeve section.

5. The liner as claimed in claim 1 wherein the electrode arrangement comprises two distal terminals which are each connected to at least one electrode, wherein the electrodes of the two distal terminals are electrically insulated from one another.

6. The liner as claimed in claim 1 wherein the protrusions on the inside of the wall are longitudinal ribs arranged in a longitudinal direction.

7. The liner as claimed in claim 1 wherein the liner is formed from a proximal sleeve part and a separate distal end part which are connected to one another by adhesive bonding and welding.

8. The liner as claimed in claim 1 wherein the liner consists of a liner shell and an insert part, wherein the insert part comprises the at least one terminal, the electrode arrangement, and at least a part of the protrusions, and wherein the insert part is insertable into the liner shell.

9. The liner as claimed in claim 8, wherein the liner shell is formed having a recess for receiving the insert part.

10. The liner as claimed in claim 1 wherein the dielectric cover of the electrode arrangement to the inside of the liner is a dielectric material which differs from material of the wall of the liner.

11. The liner as claimed in claim 1 wherein the at least one terminal for the high-voltage control signal is formed by a channel in the distal closed end section in which a contact part is insertable in a locking manner so that a central conductor of the contact part is electrically connectable to the electrode.

12. The liner as claimed in claim 11, wherein the contact part is insertable in a locking manner into the channel.

13. The liner as claimed in claim 11 further comprising a permanent magnet fastened in an electrically conductive manner on the electrode, and wherein the central conductor frontally includes a counter magnet suitable for contact on the permanent magnet in an electrically conductive connection.
